(19) United States
(12) Patent Application Publication
(10) Pub. No.: US 2022/0015758 A1
(43) Pub. Date: Jan. 20, 2022

(54) SURGICAL SUTURING DEVICE

(71) Applicant: Arkady V. Dubrovsky, Moscow (RU)

(72) Inventor: Arkady V. Dubrovsky, Moscow (RU)

(21) Appl. No.: 17/433,346

(22) PCT Filed: Feb. 13, 2020
(86) PCT No.: PCT/RU2020/000075

§ 371 Date: Aug. 24, 2021

(30) Foreign Application Priority Data
Feb. 25, 2019 (RU) 2019105155

(51) Int. Cl. A61B 17/04; A61B 17/06; A61B 17/00

(52) U.S. Cl. CPC A61B 17/0483; A61B 17/06; A61B 2017/00557; A61B 2017/06052; A61B 17/00491

(57) ABSTRACT

The suturing instrument uses a tissue suturing method consisting in introducing a quick-hardening biocompatible mass (hereinafter referred to as the ‘mass’) into the tissues to be connected or drawing it from them through hollow needles. The instrument has housing (1) with tissue gripping jaws (2 and 3) with control handles (5, 14). One of the jaws contains needle holder (19) with mechanism (17) moving it both ways. The needle holder contains mass receptacle (25, 28) above needles (18) that is connected to needle mouths via damper (33). A mechanism squeezing the mass out of the receptacle is located above the receptacle: inflatable bladder (29) that is open to external source of elevated pressure (31), rod or lock plate (59) with a device securing it to jaw walls (3) or instrument housing walls (1) on needle withdrawal from tissues. The mass receptacle presses against the fixed lock plate on needle lifting, and the mass is uniformly fed into tissues. The instrument can also be mounted on surgical forceps, (FIG. 1).

### FIELD OF THE INVENTION

The invention relates to medical equipment, and more particular it relates to a suturing instrument mainly intended for suturing biological tissues, but can also be used in other fields.

### DESCRIPTION OF PRIOR ART

Modem surgical suturing instruments containing a housing with control handles located at one end and two working jaws at the other, one fixed and the second one capable of moving relative to the first one, mainly use metal clip to suture tissues. Such clips made of alloy 40 KNKhM or of titanium are inert and biocompatible with human tissues. However, such agraffes remain a permanent foreign matter in the body.

Scar tissues develop around them as a rule. They can change their position on examination on nuclear magnetic resonance (NMR) devices.

Covidien and Ethicon suturing instruments using polylactide agraffes dissolvable in the human body after a certain period of time are known as well. (TA Premium, Poly GIA™ 75, Roticulator 55 POLY™). However, such agraffes are larger than metal ones, and are only used in gynecology and urology.

There exists a number of suturing instrument patents offering biological tissue suturing by modern synthetic dissolvable stitches using a traumatic needle or fastening elements (patents RU No. 2076639 dated Jan. 10, 1993, RU No. 2074651 dated Sep. 14, 1993, RU No. 2119771 dated Oct. 7, 1993, RU No. 2120240 dated Mar. 5, 1996, RU No. 2310405 dated Jan. 13, 2004, RU No. 2328228 dated Apr. 11, 2007, RU No. 2381756 dated Jun. 18, 2008). These instruments are more complicated than staplers, as an additional mechanism is used to fix thread ends or fastening element ends.

Two-component and multicomponent compositions instantly hardening on air or under the impact of appropriate radiation are known at present. Cobwebs are of outstanding interest. Cobweb is known to be very strong and biocompatible with human tissues and to dissolve within certain time. Many groups of researchers in Japan, USA, Canada, Sweden, China, Spain, Russia and Great Britain have created a synthetic cobweb, for example Spiber (Japan), AMSilk (Germany), Nexia Biotechnologies (Canada), Bolt Treads (California, USA), etc.

Such materials could be successfully used in surgical practice to suture biological tissues, however no suturing instrument designs suitable for use of fluid quickly as tissue fastening agents are known at present.

### CONCEPTUAL DISCLOSURE OF THE INVENTION

The task of developing a suturing instrument design adapted for use of a biocompatible fluid quick-hardening mass dissolving in human body within a certain time as a tissue fastening agent underlies this utility model.

This task is solved by using a fluid biocompatible quick-hardening mass as a fastening agent in a receptacle that is open to hollow needle channel mouths with a controllable damper mounted at the receptacle outlet and a mechanism squeezing the quick-hardening mass out of the receptacle in various types of surgical suturing instruments containing a housing with control handles at one end and two gripping working jaws at the other, at least one of which being mounted so that it can move relative to the second one, a holder with hollow needles mounted on either jaw, a hollow needle reciprocating mechanism interacting with the needle holder, as well as a tissue fastening agent and a device for feeding it into suturing needle channels according to this utility model. It is desirable to make the needle holder in the form of a rectangular hollow housing with proximal ends of hollow needles secured on its bottom. Two bladders are located inside the hollow housing. One bladder contains the quick-hardening mass and is open to hollow needle channels. The second bladder located above the first one inflates when air or liquid enters it through a special tube from a source located in the housing or outside of the instrument housing. When the upper bladder inflates, the lower bladder contents is squeezed out into hollow needles.

A single bladder divided by a partition into two isolated chambers can be used instead of two bladders, one chamber that is open to needle channels being filled with the quick-hardening mass and the second chamber, the hollow one, being open to the pressure source.

The receptacle can also be embodied as individual capsules each open to the channel of the corresponding needle. A means squeezing all capsules such as a rigid plate or bladder connected to the pressure source is located above the capsules in the needle holder cavity in that case.

Capsules individually divided by a partition into two chambers similarly to the above-described bladder can be used as well.

Another instrument embodiment can use a lock plate covering the entire surface of the quick-hardening mass receptacle(s). The plate is equipped with a locking device fixing its position relative to the instrument housing or to movable jaw walls on needle withdrawal from stitched tissues. The needle holder box bottom presses the quick-hardening mass receptacle to the fixed lock plate, and gradually squeezes its contents out of it after tissue stitching during needle lifting. The quick-hardening mass uniformly enters the stitched tissues as hollow needles leave them.

A controllable damper is mounted at the quick-hardening mass receptacle outlet. It can be a membrane rupturing as receptacle pressure rises.

The quick-hardening mass receptacle can be connected to the hollow needle mouth by soft branch pipes easily squeezed by an appropriate common damper.

The controllable damper can also be embodied as three parallel plates with through holes in a number equal to the number of hollow needles located coaxially to hollow needle channels. The middle plate is mounted so as to enable controllable travel relative to fixed extreme plates for end-to-end channel locking/opening in that case.

The proximal ends of hollow needles can also be embodied as pointed ends capable of puncturing the dampers on receptacle displacement as a result of pressure rise.

The stitching mechanism with needles and the fastening agent receptacle can be located in one working jaw, but can also be located also in different jaws.

The technical result is achieved by offering various surgical suturing instrument designs adapted for use of a quick-hardening biocompatible mass as a biological tissue fastening agent. Strong threads or mimic threads dissolving in human body are created during mass hardening. The proposed instrument embodiments enable suturing by both forcing the fluid mass through needle channels and drawing the mass through the holes formed by needles in the tissues to be sutured.

The proposed suturing instrument embodiments can be implemented on all types of surgical suturing instruments or forceps, including those intended for subcutaneous operations, and require no significant production costs. The use of quick-hardening biocompatible material dissolving within a certain time creates the most favorable prerequisites for sutured tissue healing, as the body gives no negative reaction to these materials, no foreign matter is left, and there are no nodes around which scar tissues are formed.

### BRIEF DESCRIPTION OF THE DRAWINGS

The invention is further explained by a description of its specific embodiments and the enclosed drawings:

FIG. 1 shows an embodiment of the proposed suturing instrument in general side view, partial section, the embodiment with quick-hardening mass receptacle located outside of the working jaw;

FIG. 2—is an enlarged view of working jaws, partial section;

FIG. 3—is a bottom view of hollow needle holder;

FIG. 4—is isometry of stitching mechanism;

FIG. 5—is a longitudinal section of stitching mechanism embodiment with inflatable bladder in the position before stitching;

FIG. 6—is a view same as in FIG. 5, in the initial position, before stitching;

FIG. 6a—is a view same as in FIG. 6 in the position during stitching;

FIG. 7—is a view showing needle holder in section, embodiment with capsules;

FIG. 7a—is enlarged view of two-chamber capsule in the initial position;

FIG. 7b—is a view same as in FIG. 7a at the time of stitching;

FIG. 8—is a view in closed position of a rigid damper between capsules; outlets and needle mouths;

FIG. 8a—is a view same as in FIG. 8, in open position;

FIG. 9—is a view same as in FIG. 1, but showing another suturing instrument embodiment with capsules containing the quick-hardening mass located in the movable jaw;

FIG. 10—is an enlarged view of working jaws of the suturing instrument embodiment presented in FIG. 9;

FIG. 11—is a view of suturing instrument embodiment for subcutaneous operations of the invention;

FIG. 12—is an enlarged partial section view of fixed working jaw of the instrument embodiment shown in FIG. 11 with one needle;

FIG. 13—is a view as in FIG. 12, fixed jaw embodiment with multiple needles;

FIG. 14—is a view as in FIG. 11 with quick-hardening mass capsules located in the movable jaw;

FIG. 15—is a view of surgical forceps adapted for suturing tissues by quick-hardening mass with a hollow arc-shaped spring needle and externally located quick-hardening mass receptacle;

FIG. 15a—is a view same as in FIG. 15 with quick-hardening mass entry in tissues synchronized with needle withdrawal from tissues, in the initial position;

FIG. 15b—is a view same as in FIG. 15a, at the moment of stitching termination, the quick-hardening mass receptacle is compressed, the needle is pulled out of the tissue, with the working jaws are opened;

FIG. 16—is a view same as in FIG. 15a with quick-hardening mass receptacle located in the distal half of the working jaw and fastening mass entering tissues synchronously with needle movement;

FIG. 16a—is an enlarged view of FIG. 16, distal half of the working jaw with the quick-hardening mass receptacle;

FIG. 17—is a view same as in FIG. 15 with tissue piercing needle located in one jaw, and capsules with the quick-hardening mass in the opposite one;

FIG. 18—is a view of suturing instrument embodiment with UKL type suturing instrument housing. The movable jaw houses a needle holder, a bladder with the fastening mass, and an inflatable bladder connected to an external inflatable device. Gaskets from a biocompatible fabric are located on working jaws;

FIG. 18a—is a view same as in FIG. 18 with multiple capsules containing quick-hardening mass used instead of a single bladder;

FIG. 19—is a view same as in FIG. 18 with a lock plate with a mechanism synchronizing the quick-hardening mass feeding with needle; withdrawal from the stitched tissues. The synchronization mechanism is located in the lower instrument housing section in Initial position;

FIG. 20—is a view same as in FIG. 19, showing stage of sutured tissue compression by the lowered movable jaw;

FIG. 21—is a view same as in FIG. 20, showing stage of tissue stitching by needles;

FIG. 22—is a view same as in FIG. 21, showing stage of needle pulling out of stitched tissues. The lock plate is secured, the quick-hardening mass is squeezed out of the bladder, and has entered the stitched tissues through hollow needles;

FIG. 23—is a view same as in FIG. 19 with synchronization mechanism located in the instrument housing section above the needle movement control handle;

FIG. 24—is a view same as in FIG. 19, showing longitudinal section of the movable jaw with a lock plate containing lock rods located perpendicularly to the longitudinal axis;

FIG. 25—is a view same as in FIG. 24, showing movable jaw cross section at the movable jaw rod level. Two lock rod parts pulled apart by a spring are visible in the lock plate in Initial position;

FIG. 26—is a view same as in FIG. 25, illustrating stitching stage: the needle holder is lowered, the needles have stitched the subjacent tissues. The lock rod ends have entered the movable jaw wall notches. The lock plate is secured; and

FIG. 27—is a view same as in FIG. 26, illustrating tissue stitching completion stage: the needles are lifted into the movable jaw. The bladder with quick-hardening mass is pressed to the lock plate, and the quick-hardening mass is squeezed out into the stitched tissues.

### SPECIFIC DESCRIPTION OF THE PREFERRED EMBODIMENTS

The suturing instrument of the embodiments of the invention presented on drawings include the following elements: 1—instrument housing; 2—fixed jaw; 3—movable jaw; 4—movable jaw axis; 5—movable jaw handle; 6—movable jaw handle axis; 7—handle return spring; 8—movable jaw rod pin; 9—movable jaw rod; 10—movable jaw hinge; 11—teeth on the movable jaw rod 9 end; 12—tooth lock hook; 13—lock hook spring; 14—needle positioning mechanism control handle; 15—needle positioning mechanism rod pin; 16—positioning mechanism rod, 17—needle positioning mechanism; 18—needles; 19—needle holder; 20—needle holder holes for guide pins; 21—side lugs in the needle holder; 22—guide pins for the needle holder; 23—movable plates for the needle holder, 24—slot-shaped notches in movable plates; 25—bladder with quick-hardening mass; 26—quick-hardening mass feeding hose; 27—syringe with quick-hardening mass; 28—capsule with quick-hardening mass; 28a and 29a—capsule 28 chambers; 29—inflatable bladder; 30—inflatable bladder hose; 31—external pressure source for the inflatable bladder; 32—elastic branch pipes; 33—damper; 34—damper stop; 35—damper cannulas; 36—capsules with quick-hardening mass in the movable jaw; 37—spring under capsules in the movable jaw; 38—vacuum source; 39—vacuum feed hose; 40—spring arm for the straight needle holder in an endoscopic forceps, 41—spring-loaded piston for capsules in the endoscopic forceps; 42—the first handle of the standard forceps; 43—the second handle of the standard forceps; 44—the first jaw of the standard forceps; 45—the second jaw of the standard forceps; 46—standard forceps hinge; 47—rod on the second forceps jaw; 48—rod 47 cannula; 49—rod 47 handle; 50—handle 49 return spring; 51—quick-hardening mass receptacle; 52—arc-shaped hollow needle; 53—guide hole in jaw 45; 54—groove on forceps jaw 44; 55—capsule with quick-hardening mass in forceps jaw 44; 56—spring-loaded piston in forceps jaw 44; 57—biocompatible dissolving gasket; 58—lock plate; 59—lock rod; 60—expansions on the lock rod end; 61—stops for the lock rod; 62—spring for the lock rod; 63—notch in needle holder 19 for the lock rod; 64—notch in the movable jaw wall for the lock rod.

A detailed description of suturing instrument embodiments of the invention is provided below.

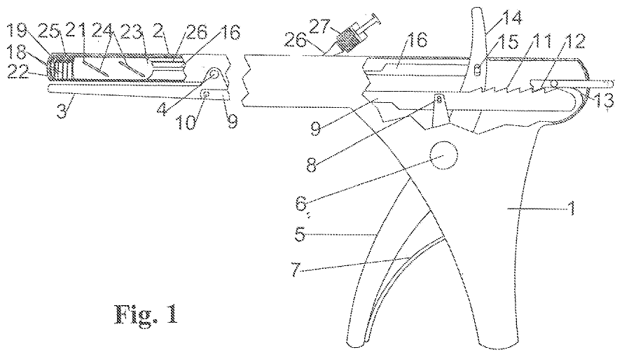

The suturing instrument presented in the general view in FIG. 1 contains housing 1 with fixed working jaw 2 and a movable jaw 3. Movable jaw 3 is mounted on the housing so that it can rotate around axis 4. Movable jaw 3 is positioned using turning handle 5 mounted in housing 1 on axis 6 and equipped with return spring 7. The short handle 5 arm has an oval hole for rod 9 pin 8. The distal end of rod 9 is connected by hinge 10 to movable jaw 3. The proximal end of rod 9 has an area with teeth 11 engaging lock hook 12 pressed by spring 13 to teeth 11. Thus, lock hook 12 retains movable jaw 3 in the specified position. Hook 12 lifting unlocks movable jaw 3.

The second movable handle 14 is mounted on housing 1 axis 6 as well to swing relative to it, and operates the needle positioning mechanism during stitching. Handle 14 has oval notch for pin 15 secured on rod 16 perpendicularly to its longitudinal axis. Rod 16 operates needle positioning mechanism 17 (FIG. 4).

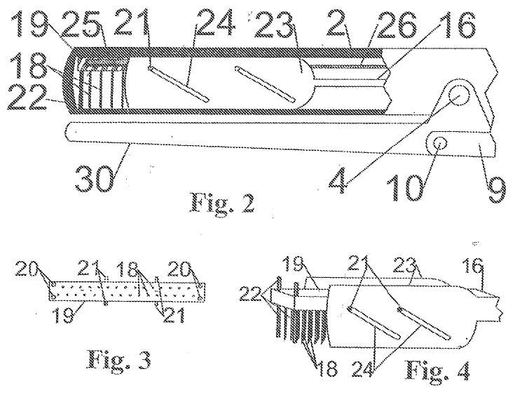

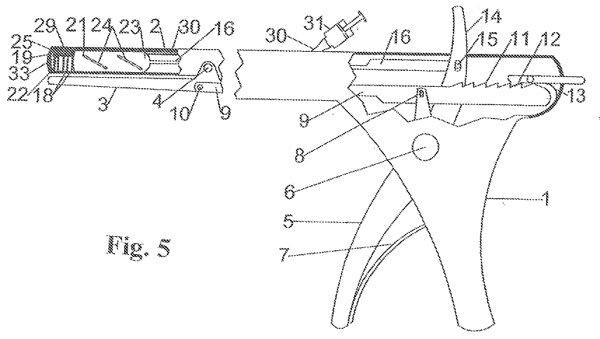

FIG. 2 is an enlarged partial section view of working jaws 2 and 3 showing several straight hollow needles 18 with upper parts secured in needle holder 19. Needle holder 19 is shown in FIGS. 3 and 4 in more detail.

FIG. 3 is a bottom view of needle holder 19 shaped as a rectangular box showing two rows of checkered holes intended to secure the proximal ends of hollow needles 18. Through holes 20 are made in holder 19 corners and side lugs 21 are present on long side surfaces as well.

FIG. 4 is an isometry of needle positioning mechanism 17. One can see that fixed guide pins 22 pass through needle holder 19 holes 20 so that holder 19 with needles 18 can perform a reciprocating sliding motion along bars 22. Bars 22 are rigidly secured in the upper and lower walls (not shown conventionally) of fixed jaw 2. Two movable plates 23 fastened to rod 16 are located in parallel on either side of needle holder 19 (FIGS. 1, 2, 4). Plates 23 can slide in the upper and lower grooves (not shown) made in the instrument jaw housing for that purpose. Plates 23 have slot-shaped notches 24 located at an acute angle to the longitudinal axis of fixed jaw 2. Holder 19 side lugs 21 with needles 18 are inserted into notch 24. This ensures that the movement of rod 16 and plates 23 causes a reciprocating movement of needle holder 19 resulting in needles 18 stitching the tissues and being pulled out of the stitched tissues.

Needle holder 19 is shaped as a hollow rectangular housing with hollow needle mouths secured in its bottom in one, two or more rows in chessboard order in this embodiment. Bladder 25 with rigid walls tightly connected to hollow needle mouths is located above the needles (FIGS. 1, 2). A damper, e.g. a membrane collapsing under elevated pressure in bladder 25 is mounted in bladder 25 joints with needles. The quick-hardening mass comes from device 27 (for example, syringe) located outside of working jaw 2 through hose 26 to bladder 25 (FIG. 1) in this embodiment. A pressure rises in syringe 27 results in pressure rise in bladder 25, membrane dampers being ruptured, and quick-hardening mass enters needle 18 channels. Bladder 25 has the minimum diameter in this embodiment.

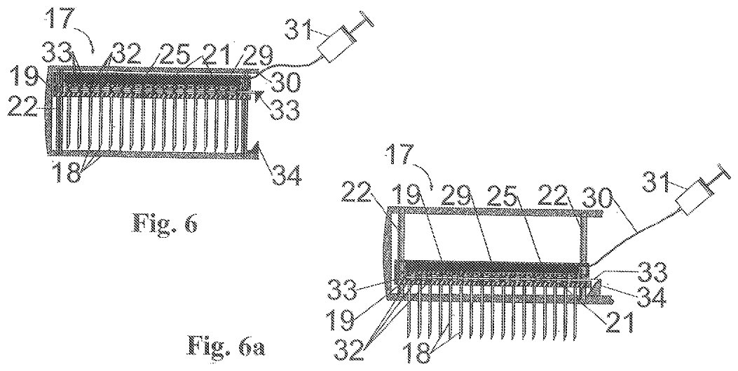

FIGS. 5, 6 and 6a show an embodiment with isolated bladder 25 with elastic walls. It is also placed in the holder 19 box and is open to hollow needle mouths 18. Inflatable bladder 29 connected by hose 30 to external pressure source 31 such as syringe and squeezing bladder 25 is located above bladder 25. Bladder 29 inflates under pressure, squeezes bladder 25. Bladder 25 is connected to hollow needle mouths 18 by elastic branch pipes 32 in this embodiment. These branch pipes are pinched by rigid damper 33 with bypasses in the initial position. The outer edge of damper 33 protrudes beyond needle holder 19 limits. Damper 33 displacement releases branch pipes 32 and opens communication between bladder 25 cavity and needle channels 18. Stop 34 mounted on fixed jaw 2 bottom is provided to displace damper 33.

FIG. 6 shows the needle positioning mechanism in the initial position: holder 19 with bladders 25 and 29 and needles 18 occupies the upper position, the outer edge of damper 33 protrudes from holder 19, damper 33 bypasses pinch branch pipes 32.

FIG. 6a shows holder 19 with bladders and needles 18 lowered almost to the lower surface of working jaw 2. The damper 33 edge touches stop 34 located on the bottom of fixed jaw 2 at this moment. When holder 19 moves further down, stop 34 displaces damper 33 with bypasses to the left, so clearing the passage in branch pipes 32. Elevated pressure in bladder 25 forces the quick-hardening mass into needle 18 channels. The preferable embodiment uses bladder 25 or capsules 28 divided by a flexible partition into two isolated chambers, one (28a) being filled with quick-hardening mass and open to needle 18 channels and the second one (29a) being hollow and open to pressure source 31. The inflatable bladder is not required in this case, which enables reducing cavity dimensions in needle 18 holder 19.

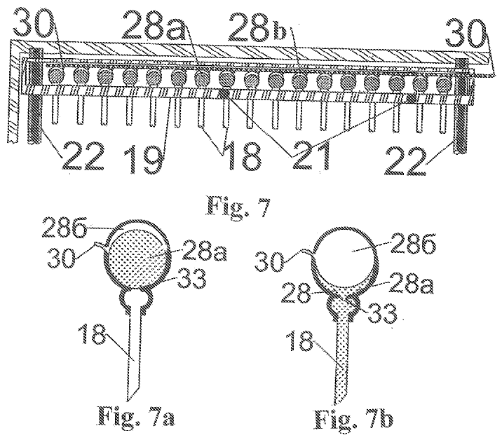

An embodiment of such instrument using capsules is shown in FIGS. 7, 7a and 7b. FIG. 7 shows that the upper parts of capsules 28 (chambers 29a) are connected to hose 30 from pressure source 31 (FIG. 5). FIG. 7a shows an enlarged section view of capsule 28 divided by a flexible partition into two chambers—lower chamber 28a filled with quick-hardening mass and open to the channel of hollow needle 18 and upper hollow chamber 29a connected by hose 30 to pressure source (FIG. 5—Syringe 31). Damper 33 in the form a thin membrane is mounted in the junction between lower chamber 28a and needle 18 channel. The damper prevents the fluid mass from early entering the needle 18 channel, but can be disrupted on capsule 28 reaching a certain pressure.

FIG. 7b shows the same capsule as in FIG. 7a in post-stitching position on upper chamber 29a pressurization from source 31. It resulted in pressure rise in chamber 28a, damper 33 destruction, and fluid quick-hardening mass entering the needle 18 channel.

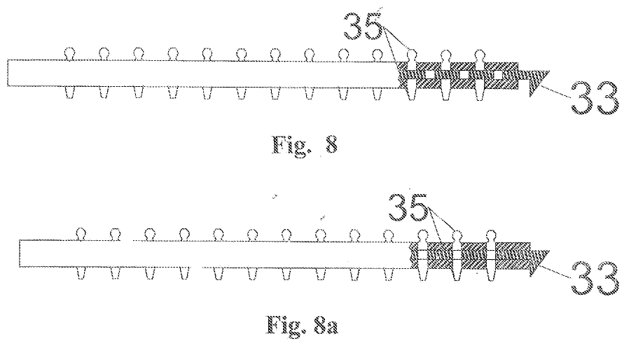

An instrument embodiment using a rigid damper is shown in FIGS. 8 and 8a. The presented design consists of three parallel plates, the middle one, that is damper 33 proper, being mounted so that it can move relative to end most fixed plates. Through holes that are coaxial to the channels of hollow needle 18 are made in the upper and lower parallel plates as well as damper 33. The hole edges of the upper plate with cannulas 35 are connected to capsules 28, and hole edges of the lower plate are connected to needle 18 channel mouths, respectively, in that case. The displacement of damper 33 blocks or releases the communication between needle 18 channels and capsules 28. The configuration of the upper and lower cannulas 35 can vary to create a tight connection.

FIG. 8a shows damper 33 displaced to the left to release quick-hardening mass passage to hollow needle 18 channels.

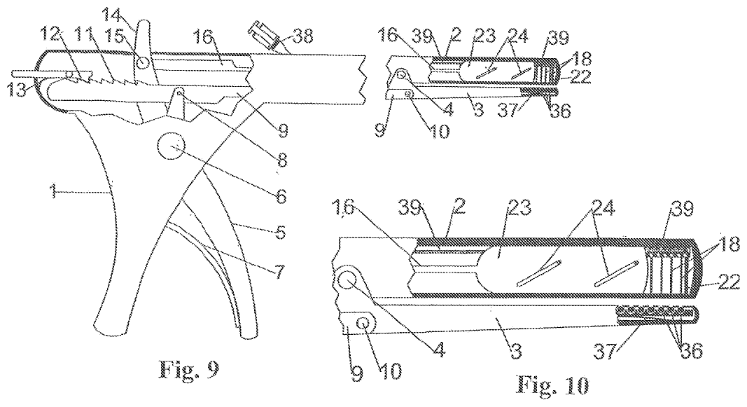

FIGS. 9 and 10 show another instrument embodiment where capsules 36 with quick-hardening mass are located in movable jaw 3. The positioning mechanism with needles 18 is mounted in fixed jaw 2. Needles 18 stitch tissues, their sharp ends enter jaw 3, and puncture capsules 36 in this embodiment. Then the needles are slowly extracted from tissues, and quick-hardening mass from capsules 36 enters tissues through punctures after needles to fill the space remaining in tissues after needle extraction. A process of ‘drawing’ the quick-hardening mass from punctured capsules 36 takes place. The process can be accelerated by creating elevated pressure in capsules 36 and creating vacuum in the puncturing hollow needles. Spring 37 is located under capsules to create elevated pressure in capsules 36. Appropriate pipes connect the proximal ends of hollow needles to vacuum source 38 (for example, syringe 38, see FIG. 9) to create vacuum in hollow needle channels. This instrument embodiment can be used to suture fine tissues. The instrument for suturing biological tissue by quick-hardening mass can be also mounted on the forceps used in subcutaneous operations.

FIGS. 11-16 show these embodiments. FIG. 11 shows a surgical device configured as an endoscopic forceps containing housing 1 with a fixed handle in the proximal section. The distal section of the housing contains fixed working jaw 2. The second working jaw 3 can move relative to fixed jaw 2 and is mounted on axis 4. Jaw 3 is connected to movable handle 5 rotating on axis 6. The small handle 5 arm is connected by pin 8 to rod 9. The distal end of rod 9 is connected to jaw 3 by hinge 10. The fixed and movable handles bear rings for the surgeon's fingers at proximal ends and rack-and-pinion gear enabling fixing the position of movable handle 5 and movable jaw 3. Housing 1 also contains spring-loaded handle 14 moving rod 16. Rod 16 is located inside housing 1 and fixed jaw 2 and moves holder 19 with hollow needle 18. External pressure source 31 with hose 30 is located slightly below handle 14.

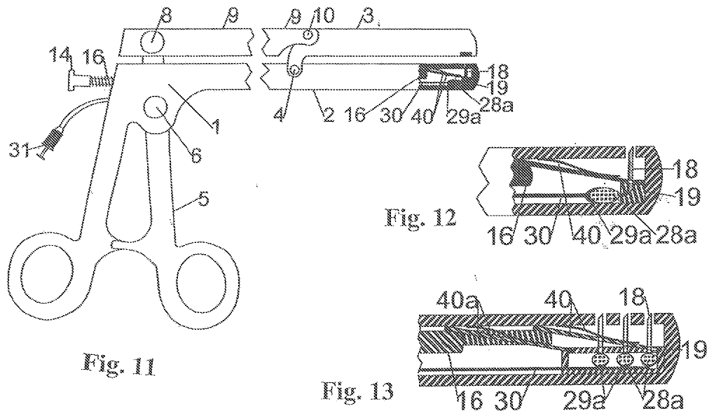

FIG. 12 is a slightly enlarged section view of the distal end of jaw 2. Hollow needle 18 is secured in holder 19. The proximal end of needle 18 is bent to reduce the jaw diameter and connected to two-chamber capsule 28. A part of capsule 28, chamber 28a, is filled with quick-hardening mass and connected through membrane damper to hollow needle 18 mouth. The second part of capsule 28, chamber 29a, is hollow, and is connected by hose 30 to external pressure source 31. Holder 19 is mounted in the fixed jaw 2 housing, slides in special notches in the end wall and side walls of this jaw and is connected to lever 40. Lever 40 is located at an acute angle to jaw 2 surface adjoining the tissues to be stitched.

The distal end of lever 40 is secured to axis on holder 19 housing, and the proximal end of lever 40 is secured on axis on the internal surface of jaw 2 side walls. A spring is located between lever 40 and jaw 2 working surface. The distal end of rod 16 with raked surface adjoins lever 40 without side. If pressure is applied to handle 14, then rod 16 presses lever 40 together with holder 19 to jaw 2 working surface. Needle 18 secured in holder 19 stitches the tissues in that case. Elevated pressure is applied from source 31. Inflatable chamber 29a begins to squeeze the quick-hardening mass out of chamber 28a into the stitched tissues. Handle 14 is released, rod 16 returns to the initial position. The lever 40 spring returns lever 40 and holder 19 with needle 18 to the initial position as well.

FIG. 13 shows the same embodiment as in FIG. 12, but with multiple needles—three needles 18 in this case. Double spring-loaded lever 40 and 40a is provided in this embodiment in accordance with dimensions of holder 19 with multiple needles 18.

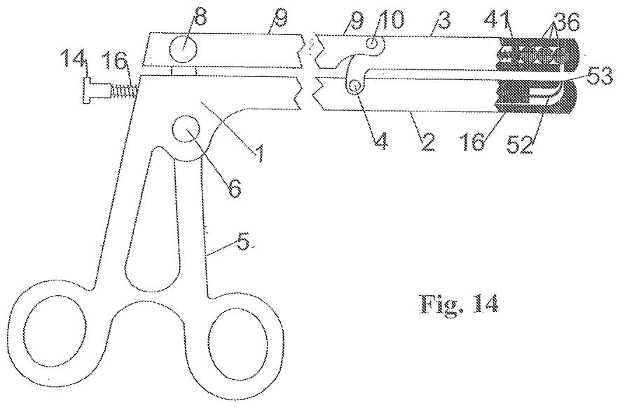

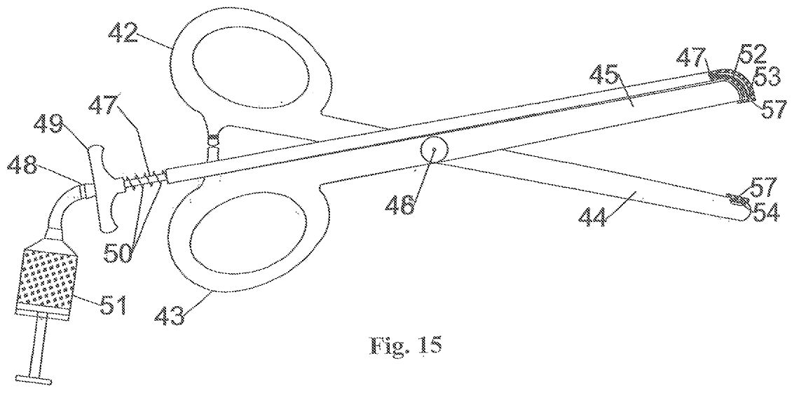

FIG. 14 shows another embodiment of the suturing instrument configured as an endoscopic forceps. Needle 18 is located in fixed working jaw 2, and capsules 36 with quick-hardening mass are located in the opposite movable jaw 3 in this embodiment. Slightly springing arc-shaped needle 52 secured in the distal end of rod 16 can be used in this embodiment. The sharp end of needle 52 is located in rigid guide hole 53 in the jaw 2 working surface adjoining the tissues to be stitched. This hole ensures a relatively rectilinear needle end movement in the space between squeezed jaws 2 and 3 and needle end entry in the appropriate hole in jaw 3. Needle end punctures capsule 36 with quick-hardening mass in jaw 3. Spring-loaded handle 14 is released, and the needle returns to the initial position in jaw 2 while drawing the quick-hardening mass from punctured capsule 36. One capsule 36 can be located in jaw 3, but two or more capsules with quick-hardening mass can be located as well.

Spring-loaded piston 41 accelerating the quick-hardening mass efflux from punctured capsule 36 into tissues squeezed by jaws as well as moving spent capsules 36 is mounted behind the capsules.

Similar instruments forcing and drawing the quick-hardening mass can be mounted also on standard surgical forceps as well to enable their use as suturing instruments.

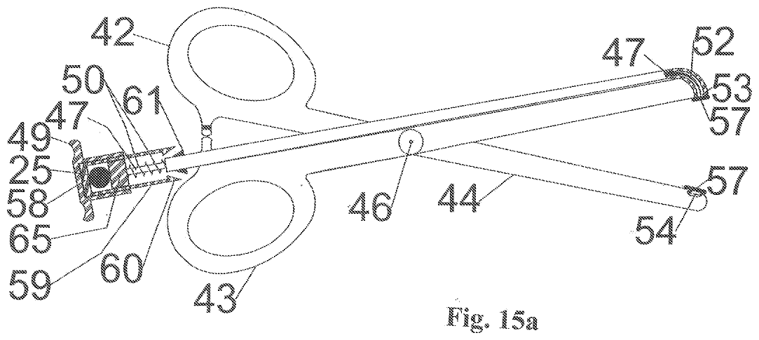

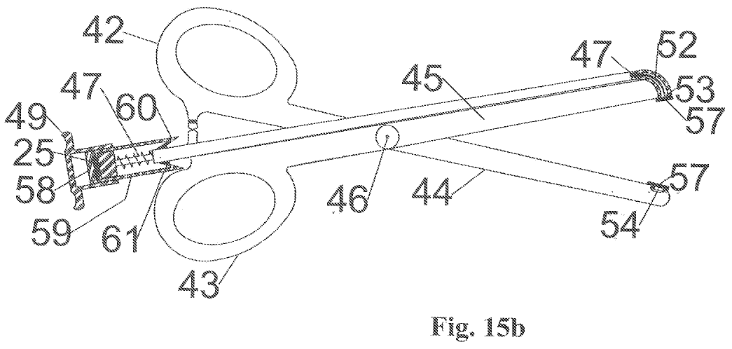

FIG. 15 shows a standard surgical forceps consisting of two identical parts with handles 42 and 43 and working jaws 44 and 45 connected by hinge 46. Hollow rod 47 in a special casing is mounted either part of the forceps. Handle 49 with cannula 48 is located on the proximal end of rod 47. Return spring 50 is mounted in front of handle 49 on rod 47. Receptacle 51 with quick-hardening mass (for example, a syringe) is inserted into cannula 48. Hollow arc-shaped springing needle 52 is secured to the distal end of hollow rod 47. The sharp end of needle 52 is located in guide hole 53 at the end of jaw 45. Small groove 54 receiving the end of needle 52 after tissue puncturing is located at the distal end of jaw 44. It is desirable to cover the surfaces of both jaws adjoining the tissues to be stitched in the area of needle puncture with gaskets from biocompatible dissolving materials. These gaskets 57 are shown in FIG. 15.

If pressure is applied to handle 49, then rod 47 pushes needle 52 through hole 53. Needle 52 punctures tissue squeezed by working jaws 44 and 45 through gasket 57, and enters groove 54 through the second gasket 57. Then quick-hardening mass is fed from receptacle 51 to fill groove 54 and impregnates gasket 57. Then the mass follows the needle to uniformly enter the tissues to be sutured, and fills the space in the punctured tissues during gradual needle 52 withdrawal from tissues. This mass quickly hardens and fastens the stitched tissues. Gasket 57 increases the suture tightness and reliability.

The synchronism of needle withdrawal from tissue and fastening mass entry in tissues is extremely important in this process. An instrument embodiment ensuring this synchronism is therefore proposed. This embodiment is shown in FIGS. 15a and 15b. The design of this embodiment in the initial position is shown in FIG. 15a. Handle 49 is shaped as a hollow cylinder. Hollow rod 47 is secured in the cylinder bottom. The proximal end of rod 47 is located inside the cylinder and is pointed. Capsule 25 with quick-hardening mass is located inside the cylinder. The mouth of capsule 25 with damper is connected to the pointed end of rod 47. Lock plate 58 with two (or more) lock rods 59 is located in front of capsule 25. Lock rods 59 freely move in corresponding holes in the bottom of cylindrical handle 49 in parallel to rod 47. The lock rods ends can be springy, and triangular expansions 60 are present on them.

The lock rods press against the hollow rod 47 casing with triangular expansions 61 of opposite orientation on its walls. If pressure is applied to handle 49, then hollow rod 47 pushes hollow arc-shaped needle 52 into the tissues to be stitched. The movement of handle 49 causes lock rods 59 to move along rod 47 casing. The expansions on the ends of lock rods 59 engage expansions 61 on rod 47 casing at the final stage of handle 49 movement when needle 52 has punctured the tissues. It fixes lock plate 58. When handle 49 is released, spring 50 returns handle 49 and rod 47 to the initial position. Bladder 25 presses against fixed lock plate 58. The pointed proximal end of rod 47 punctures the bladder 25 damper, and quick-hardening masses from bladder 25 enter the stitched tissues through hollow rod 47 and needle 52 synchronously with needle 52 withdrawal from the tissues. The final moment of stitching is shown in FIG. 15b: needle 52 is withdrawn from the tissue, bladder 25 is completely compressed, and quick-hardening masses are completely squeezed out into the tissues.

This instrument can be located closer to the distal end of the corresponding forces jaw to save the quick-hardening mass.

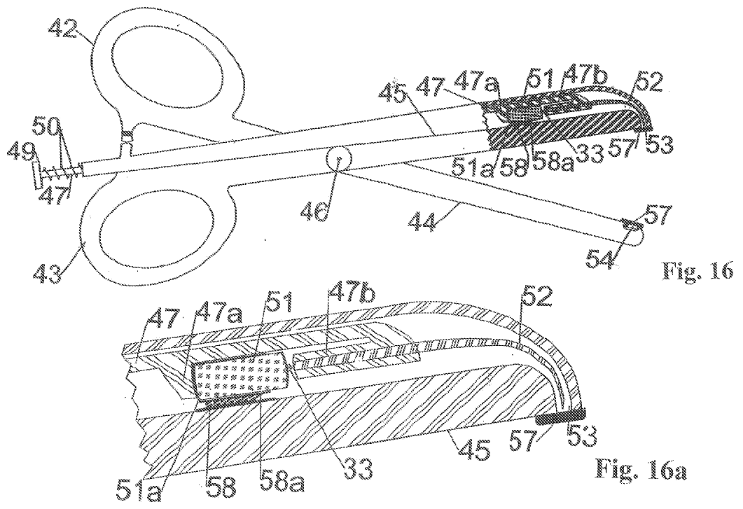

This embodiment is shown in FIGS. 16 and 16a. FIG. 16 shows slightly expanded rod 47 casing in the distal half of jaw 45 (this part of jaw 45 is shown in FIG. 16a in enlarged view). Oblong quick-hardening mass receptacle 51 is located in this area under rod 47. Receptacle 51 is located in cylindrical casing 51a that is open on the distal side. The lower surface of casing 51a is covered by triangular lugs with pointed ends looking in the distal direction (58a). This surface adjoins lock plate 58 secured onto forceps jaw 45. Plate 58 also has triangular lugs with pointed ends looking in the proximal direction. The interaction of scalloped surfaces 58 and 58a will enable the movement of cylindrical casing 51a with receptacle 51 in the distal direction alone. Rod 47 has two lugs bracing receptacle 51 on both sides. Lug 47a adjoins to the proximal end of cylinder 51a and moves cylinder 51a with receptacle 51 when rod 47 moves in the distal direction. Profiled lug 47b of diameter equal to receptacle 51 diameter adjoins receptacle 51 at the distal end. Hollow needle 52 is secured in this lug. The proximal end of needle 52 is pointed, slightly protrudes from lug 47b, and adjoins damper 33 located at the distal end of receptacle 51. Rod 47 advances needle 52 into tissues, and lug 47a simultaneously moves receptacle 51 in the distal direction on tissue stitching by squeezed forceps jaws. When rod 47 moves in the reverse direction, needle 52 is withdrawn from tissues, lug 47b enters the pointed proximal end of needle 52 into damper 33, and lug 47b gradually squeezes receptacle 51, as receptacle 51 casing is fixed by lock plates 58 and 58a. The receptacle 51 contents enter needle 52, and then the stitched tissue synchronously with needle 52 withdrawal from the tissues.

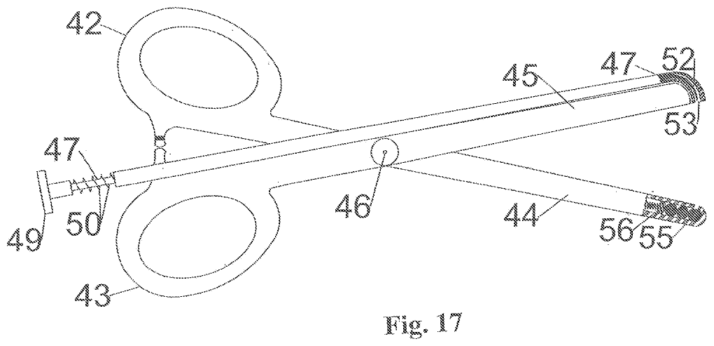

FIG. 17 shows another embodiment of a surgical forceps adapted for stitching tissues drawing a quick-hardening mass. Rod 47 ending with handle 49 with return spring 50 at the proximal end is mounted on jaw 45 in this embodiment as in FIG. 15. Arc-shaped springing needle 52 with sharp end guided by hole 53 during stitching is located at the distal end of rod 47. Capsule(s) 55 with quick-hardening mass is located in the opposite jaw 44 and is spring-loaded by piston 56. Once the tissues to be sutured are squeezed by jaws 44 and 45, pressure is applied to handle 49, and rod 47 pushes needle 52 from jaw 45. Once tissues are stitched, the needle enters jaw 44, and punctures the adjacent capsule 55. Spring 50 returns needle 52 into jaw 45. The quick-hardening mass flows from punctured capsule 55, and fills the space in punctured tissues after leaving needle to fasten the tissues together. Spring-loaded piston 56 accelerates this efflux. Rod 47 and needle 52 can be solid, but can also be hollow as in FIG. 15 in this embodiment. A syringe can be connected to the proximal end of rod 47 (as in FIG. 15) to create vacuum in needle 52, which will additionally accelerate the quick-hardening mass entry in the tissues to be sutured.

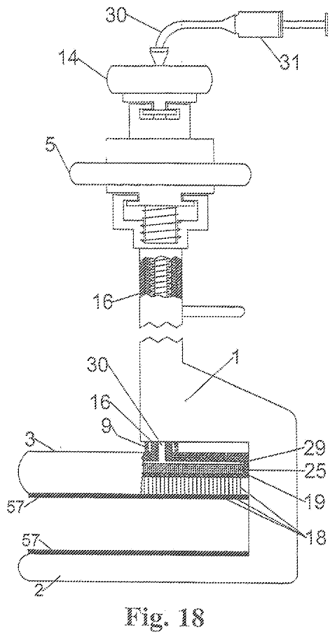

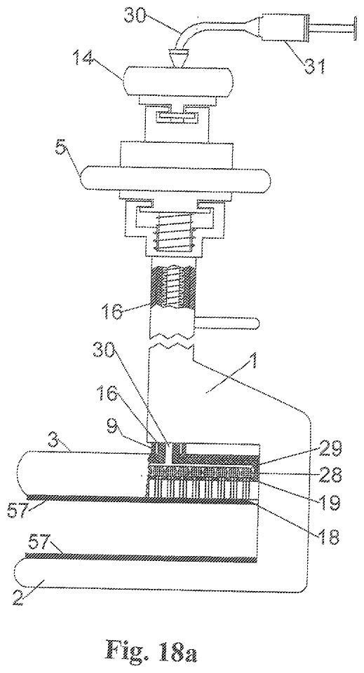

The suturing instrument can also be mounted on the basis of a known UKL type suturing instrument. Such instruments are shown in FIGS. 18 and 18a.

FIG. 18 shows an instrument with housing 1 and fixed working jaw 2 located perpendicularly to the longitudinal axis of the housing. Movable working jaw 3 is located in parallel to fixed jaw 2. The handle of movable jaw 5 is located in the proximal section of housing 1 and is connected (threaded connection) to movable jaw 3 by hollow rod 9 located inside housing 1. The rotation of handle 5 causes a linear displacement of rod 9 and movable jaw 3. Handle 14 controlling the movement of needles 18 through rod 16 is located above handle 5. Rod 16 is hollow too, is located inside rod 9, and is connected to needle holder 19. Needle holder 19 is a rectangular hollow box located in movable jaw 3. Hollow needles 18 are secured in holder 19 bottom in one, two or more rows in chessboard order. Bladder 25 with quick-hardening mass is located inside holder 19 above needle mouths. Bladder 25 is connected to needle mouths by damper 33 (not shown in the Figure, as it can be one of the above-described damper embodiments). Inflatable bladder 29 connected by hose 30 to external pressure source 31 is located above bladder 25. Hose 30 passes inside hollow rod 16, but a part of it is located above handle 14.

It is desirable to place gaskets 57 from biocompatible tissues dissolving within a certain time on the surfaces of jaws 2 and 3 adjoining the tissues to be stitched. These gaskets increase the joint tightness and reliability. Thus, the rotation of handle 5 causes movable jaw 3 with needles and their positioning mechanism to lower. The tissue to be sutured are compressed. The rotation of handle 14 lowers needle holder 19 in the movable jaw, and needles 18 puncture the compressed tissues through gaskets 57. Air/liquid supply starts from instrument 31 into inflatable bladder 29 at this moment. Bladder 25 is squeezed, and quick-hardening mass from bladder 25 begins to come into the tissues to be sutured through hollow needles. When handle 14 rotates in reverse direction, the needles are withdrawn from tissues simultaneously with quick-hardening mass entry in the tissues to be sutured. The tissues to be sutured are connected by forming threads.

FIG. 18a shows the same embodiment, but bladder 25 in holder 19 is replaced with separate capsules 28 with the quick-hardening mass. The feed of quick-hardening mass from bladder 25 or capsules 28 can be synchronized with the moment of needle withdrawal from tissues.

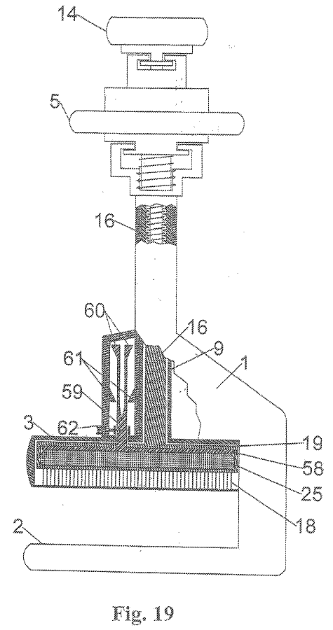

FIGS. 19, 20, 21 and 22 show one of synchronization options and its consecutive stages. Rigid lock plate 58 is located in the cavity of rectangular needle holder 19 box on the upper surface of bladder 25 or capsules 28 with quick-hardening mass instead of inflatable bladder 28.

Plate 58 is equipped with a locking device, rod 59, enabling plate 58 motion in the needle holder 19 box only in one direction. Lock rod 59 is secured onto plate 58 or is located inside it. The second end of rod 59 is fixed in a certain point on the instrument housing or in the movable jaw wall. Plate 58 goes down into movable jaw 3 cavities together with needle holder 19 in the course of tissue stitching, but when it reaches a certain level, and needle lifting starts, lock rod(s) 59 firmly secures plate 58 in this position relative to instrument 1 housing or movable jaw 3 walls.

Needle holder 19 bottom squeezes bladder 25 (or capsules 28) by pressing it to fixed lock plate 58 on needle lifting. The quick-hardening mass enters hollow needles 18 and stitched tissues synchronously with needle withdrawal from tissues, as the volume of mass entering the tissues precisely matches the needle lifting level.

FIG. 19 shows a locking device embodiment. Rod 59 is secured to the upper surface of plate 58 in parallel to rod 16. The upper part of rod 59 is located in the slightly expanded lower part of housing 1. The upper part of rod 59 is split, and ends with triangular expansions 60 with broad part looking upwards. The splitting of the upper part of the rod enables rod 59 end springing. Triangular expansions 61 with broad part looking upwards are also present at a certain level on walls of the cavity where this part of the rod is located. The combination of these expansions enables rod 59 displacement to one side only. FIG. 19 shows the starting position when working jaws 2 and 3 are pulled apart.

FIG. 20 shows the moment of squeezing the tissues to be stitched: movable jaw 3 is lowered by handle 5. The locking device 59 rod has lowered itself in the lower part of housing 1 as well.

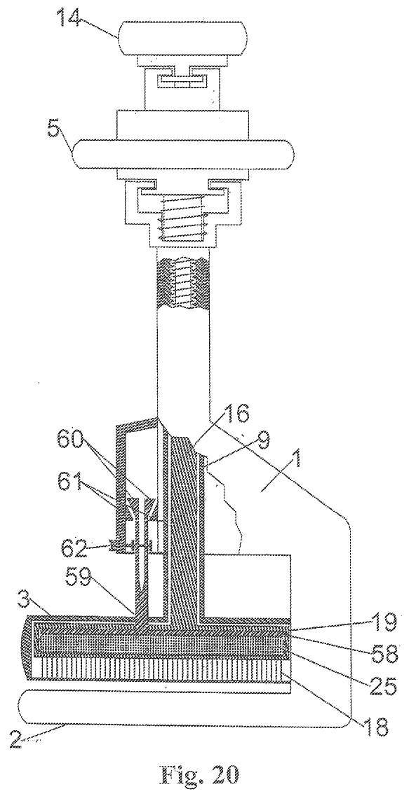

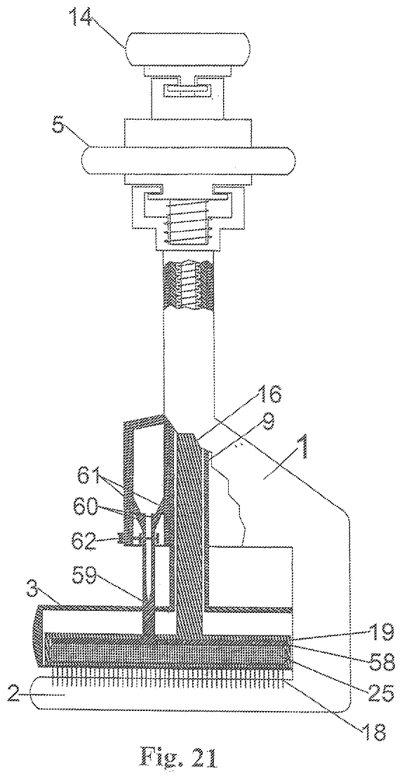

FIG. 21 shows the moment of stitching: handle 14 and rod 16 lower down needle holder 19 into movable jaw 3 cavities. Needles 18 stitch the tissues squeezed by jaws 2 and 3. Rod 59 goes down together with holder 19. Upper springing ends 60 of rod 59 pass the narrowed area created by triangular expansions 61. The reverse motion of rod 59 is excluded. Triangular expansions 60 and 61 enable rod 59 motion only in one direction only.

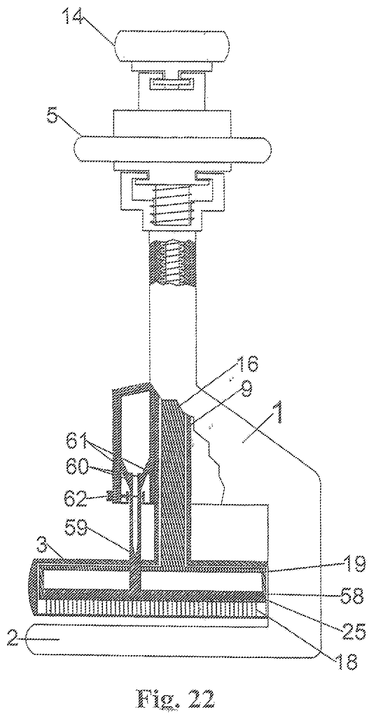

FIG. 22 shows needle 18 withdrawal from stitched tissues. When handle 14 moves in reverse direction, needle holder 19 rises in movable jaw 3, and withdraws needles 18 from the stitched tissues. However, rod 59 with plate 58 are secured at the lower level. The needle holder 19 box bottom presses bladder 25 or capsules 28 to fixed lock plate 58, and squeezes the quick-hardening mass out of them. It enters the hollow needles and stitched tissues synchronously with needle withdrawal from tissues. When handle 62 turns, springing expanded ends 60 of rod 59 are compressed, and freely pass between expansions 61. Movable jaw 3 rises, and the suturing instrument is removed.

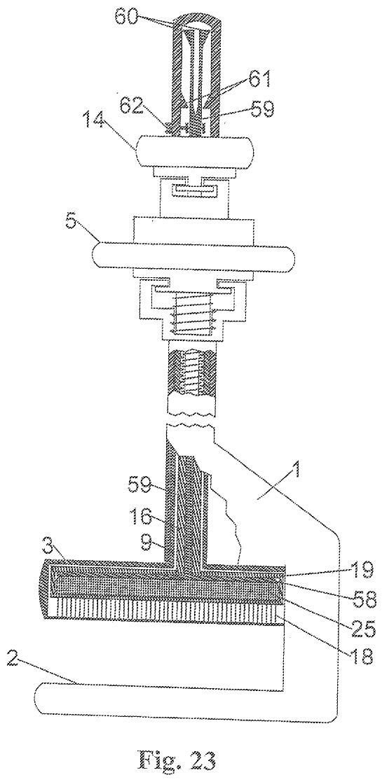

FIG. 23 shows a different locking device arrangement, above handle 14. Rod 59 is located inside rod 16 in this embodiment.

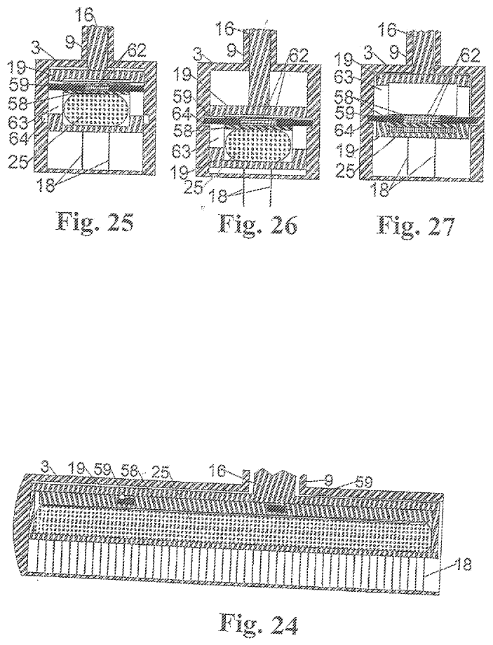

FIGS. 24, 25, 26 and 27 shows another locking device embodiment. Spring-loaded lock rods 59 are located inside lock plate 58 along long axis or across it in this embodiment. Each rod 59 is divided into two parts with spring 62 between them. The peripheral ends of lock rods which are pushed out by the spring are secured in the corresponding slots located in movable jaw 3 walls. Special notches 63 designed for free motion of lock rods 59 inside them should be present in needle holder 19 box walls.

FIG. 24 shows movable jaw 3 in section along its long axis. Lock plate 58 is located above bladder 25 with quick-hardening mass. Two lock rods 59 are located inside plate 58 perpendicularly to its longitudinal axis.

FIG. 25 shows the cross section of movable jaw 3 at the level of one of lock rods 59 located under rod 16. Lock rod 59 is located inside plate 58 and consists of two parts with spring 62 between them in this embodiment. The peripheral ends of lock rods 59 are located in needle holder 19 box wall notches 63 and abut against movable jaw 3 walls. Notches 64 for lock rod 59 ends are available at a certain level in movable jaw 3 walls. FIG. 25 shows the initial position where needle holder 19 with needles 18 and bladder 25 is located in the upper section of movable jaw 3. The ends of spring-loaded rods 59 are located in needle holder 19 box wall notches 63, and abut against movable jaw 3 housing walls.

FIG. 26 shows the same cross section of movable jaw 3 on tissue stitching: rod 16 lowers needle holder 19 down in movable jaw 3, and needles 18 stitch the tissues. The ends of lock rods 59 pulled apart by spring 62 enter notches 64 in movable jaw 3 walls at this moment. They firmly secure plate 58 relative to movable jaw 3 housing.

FIG. 27 shows the final moment of stitching: rod 16 lifts needle holder 19 in the movable jaw 3 cavity. Needles 18 are withdrawn from tissues. The needle holder 19 box bottom presses bladder 25 to fixed plate 59, and squeezes quick-hardening masses out of it into hollow needles.

The stitching is completed. The quick-hardening mass enters the punctured tissues synchronously with needle withdrawal from tissues.

Thus, suturing instrument design embodiments using biocompatible quick-hardening materials in various types of suturing instruments and surgical forceps are shown. It enables creating the most favorable conditions for healing of the tissues to be sutured as well as will facilitate and speed up surgeries. https://www1.fips.ru/registers-doc-view/DeEndhttps://www1.fips.ru/registers-doc-View/CIStart

1. A surgical suturing device comprising, a housing with control handles at one end and two gripping working jaws at the other, at least one of which being mounted so that it can move relative to the second one, a holder with hollow needles mounted on either jaw, a reciprocating mechanism for hollow needle mounted so that they can interact with the needle holder, as well as a tissue fastening agent, wherein a fluid biocompatible quick-hardening mass placed in a receptacle open to hollow needle channel mouths and equipped with a mechanism ensuring its uniform feed into the tissues to be stitched is used as a fastening agent, and a damper mounted at the receptacle outlet.

2. A suturing device of claim 1, wherein the mechanism for uniform feeding of fluid mass contains a lock plate located close to the tissue fastening agent receptacle with a device securing the plate to movable jaw or instrument housing walls after tissue puncturing by needles, the hollow needle reciprocating mechanism squeezing the tissue fastening agent receptacle adjoining the fixed lock plate synchronously with needle withdrawal from tissues.

3. A suturing device of claim 1, wherein the tissue fastening agent receptacle is divided by a partition into two isolated chambers, one being open to needle channels and filled with quick-hardening mass and the other being hollow and open to a pressure source as well as performing the function of a mechanism uniformly feeding the fluid mass on pressure application.

4. A suturing device of claim 1, wherein the needle holder is shaped as a straight hollow housing, the tissue fastening agent receptacle is embodied at a set of capsules in a number equal to the number of suturing needles each open to channel of the corresponding needle, with a device squeezing all capsules and connected to a pressure source being additionally located in the holder cavity.

5. A suturing device of claim 1, wherein the damper is made as a membrane collapsing under elevated pressure in the tissue fastening agent receptacle.

6. A suturing device of claim 1, wherein the proximal ends of hollow needles are pointed and embodies so that the damper could be punctured on displacement as result of pressure rise in the tissue fastening agent receptacle.

7. A suturing device of claim 1, wherein the needle holder is shaped as a straight hollow housing and the receptacle is embodied as a bladder from elastic material placed inside the holder, the bladder being connected to needle channels by elastic branch pipes.

8. A suturing device of claim 1, wherein the damper is embodied as three parallel plates with through holes in a number equal to the number of hollow needles located coaxially to hollow needle channels, the middle plate being mounted so that it can move relative to end most fixed plates and block through channels.

9. A suturing device of claim 1, wherein the stitching mechanism with needles and tissue fastening agent receptacle are located in one working jaw.

10. A suturing device of claim 1, wherein the stitching mechanism with needles and fastening agent receptacle are located in different working sponges.
